(19) United States
(12) Patent Application Publication
(10) Pub. No.: US 2023/0185524 A1
(43) Pub. Date: Jun. 15, 2023

(54) SYSTEM AND METHOD FOR LEVERAGING AUDIO COMMUNICATION AND GUIDANCE TO IMPROVE MEDICAL WORKFLOW

(71) Applicant: KONINKLIJKE PHILIPS N.V., EINDHOVEN (NL)

(72) Inventors: Ekin KOKER, CAMBRIDGE, MA (US); Olga STAROBINETS, NEWTON, MA (US); Christian FINDEKLEE, NORDERSTEDT (DE); Siva Chaitanya CHADUVULA, MALDEN, MA (US); Ranjith Naveen TELLIS, TEWKSBURY, MA (US); Sandeep Madhukar DALAL, WINCHESTER, MA (US); Falk UHLEMANN, NORDERSTEDT (DE); Qianxi LI, CAMBRIDGE, MA (US); Thomas Erik AMTHOR, HAMBURG (DE); Yuechen QIAN, LEXINGTON, MA (US)

(21) Appl. No.: 17/994,477

(22) Filed: Nov. 28, 2022

Related U.S. Application Data
(60) Provisional application No. 63/289,699, filed on Dec. 15, 2021.

(51) Int. Cl. G06F 3/16; G10L 15/22

(52) U.S. Cl. CPC G06F 3/167; G10L 15/22

(57) ABSTRACT

A communication system between an imaging bay containing a medical imaging device and a control room containing a controller for controlling the medical imaging device includes an intercom with a bay audio speaker and bay microphone in the imaging bay, and a communication path via which bay audio from the bay microphone is transmitted to the control room and via which instructions are transmitted from the control room to the bay audio speaker. An electronic processing device operatively connected with the communication path is programmed to at least one of (i) generate the instructions; (ii) modify the bay audio and output the modified bay audio in the control room; and/or (iii) analyze the bay audio to determine actionable information, determine a modification of or addition to a medical workflow based on the actionable information, and automatically implement the modification of or addition to the medical workflow.

### CROSS-REFERENCE TO RELATED APPLICATIONS

This application claims the benefit of U.S. Provisional Application No. 63/289,699 filed Dec. 15, 2021, the specification of which is incorporated herein by reference in its entirety.

The following relates generally to the medical imaging arts, remote imaging assistance arts, and related arts.

### BACKGROUND

Electronic audio communication is commonly used in medical workflows. Current medical imaging workflows for example require imaging technologists to handle patient activities and guide them through the imaging workflow. Communication between the imaging technologist and the patient is a key component to achieving a successful imaging examination. In many types of imaging examinations, the patient will receive verbal instructions from the imaging technologist, and image quality is dependent upon the patient understanding and following these instructions. For example, in some imaging examinations the patient may be instructed to perform a breath-hold during image acquisition to avoid image blurring due to patient respiration, or more generally may be instructed to remain still during image acquisition. In some types of contrast enhanced imaging, the patient may be administered an intravascular contrast agent at a certain point during the examination, and the patient may receive instructions pertaining to that operation. Communication in the opposite direction, that is, verbal communication from the patient to the imaging technologist, can also be critical. If the patient experiences pain, claustrophobia, or other discomfort, this should be clearly and immediately conveyed to the imaging technologist so that appropriate remedial action can be performed. If the patient does not speak the same language as the technologist, this further complicates the communication, as an additional translator is also needed. Communication from the patient can be difficult to understand due to the large amount of noise generated by certain imaging modalities such as magnetic resonance imaging (MRI) and the placement of the patient in an enclosed imaging bore in some imaging modalities.

Where translator services are not available as easily or are expensive, patient instructions in the more common foreign languages for the patients served may be made available in writing with words phonetically spelled out for technologists to use as a low cost communication option. However, this method is inflexible and can lead to misunderstandings if the patient needs clarification on the instructions, and can pose safety issues if the patient tries to communicate back to the technologist and the technologist does not speak the same language. The language barrier can be even greater when both the imaging technologist and the patient can communicate in a common language, but that common language is a second language for one or both of them. For example, if the patient speaks the technologist’s native language as a second language with something less than full fluency, then the patient has an increased likelihood of misunderstanding the technologist due to the patient’s weak command of the language. These various communication problems can be further aggravated by the technical domain of medical imaging which can lead the technologist to use terms that may be unfamiliar to the patient.

A recent development in the medical imaging field is the use of remote experts to assist a local imaging technician during a challenging imaging examination. In some scenarios, the remote expert could be located in a different region or even a different country. This introduces the possibility that each of the three actors: remote expert, local imaging technician, and patient, may speak different languages, or may have varying levels of fluency in a common language.

Another example of the use of electronic audio communication is in the area of telehealth, i.e. providing of medical care to a patient from a remote location via a telephonic or video call or the like. Telehealth and virtual care have gained significant traction over the last few years. One challenge of telemedicine is to ensure that the quality of care does not suffer when delivered in a virtual setting. However, present day face-to-face physician-patient interactions involve personal discussions and probing questions that can lead the physician to recognize patient issues that are not directly verbalized. Such unexpected revelations are likely to suffer when done virtually.

Early identification of the patient’s health status may also serve as information for scheduling since it may help to predict the exam time demanded for each individual patient.

The following discloses certain improvements to overcome these problems and others.

### SUMMARY

In one aspect, a communication system for communicating between an imaging bay containing a medical imaging device and a control room containing a controller for controlling the medical imaging device. The communication system includes an intercom including a bay audio speaker disposed in the imaging bay, a bay microphone disposed in the imaging bay, and a communication path via which bay audio from the imaging bay acquired by the bay microphone is transmitted to the control room and via which instructions are transmitted from the control room to the bay audio speaker for output by the bay audio speaker; and an electronic processing device operatively connected with the communication path and programmed to at least one of (i) generate the instructions; and/or (ii) modify the bay audio and output the modified bay audio in the control room.

In another aspect, a communication method for communicating between an imaging bay contains a medical imaging device and a control room containing a controller for controlling the medical imaging device. The communication method includes receiving bay audio from the imaging bay at the control room; using an electronic processing device, modifying the bay audio to generate modified bay audio; and presenting the modified bay audio in the control room.

In another aspect, a non-transitory computer readable medium stores instructions executable by at least one electronic processor to perform a communication method for communicating between an imaging bay containing a medical imaging device and a control room containing a controller for controlling the medical imaging device includes using an electronic processing device, generating instructions for a patient in the imaging bay; transmitting the instructions to the control room; and presenting the transmitted instructions to the patient in the imaging bay.

One advantage resides in determining a state of a patient during an imaging examination.

Another advantage resides in determining a state of a patient during an imaging examination by analyzing vocals of a patient.

Another advantage resides in determining a state of a patient during an imaging examination by analyzing biomarkers of a patient.

Another advantage resides in determining a state of a technologist during an imaging examination.

Another advantage resides in providing improved communication between the patient and the imaging technician during a medical imaging examination.

Another advantage resides in providing translation of language of communications between a patient and one or more medical professionals.

Another advantage resides in leveraging an existing intercom system to provide translation of language of communications between a patient and one or more medical professionals.

Another advantage resides in providing sign language translation services between a patient and one or more medical professionals.

Another advantage resides in reducing complexity of language in communications between a patient and one or more medical professionals.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 diagrammatically shows an illustrative apparatus for performing a communication method in accordance with the present disclosure.

FIG. 2 shows example flow charts of operations suitably performed by the apparatus of FIG. 1.

FIG. 3 shows another example of operations suitably performed by the apparatus of FIG. 1.

FIG. 4 shows another example flow chart of operations suitably performed by the apparatus of FIG. 1.

FIG. 5 shows a generalized example flow chart of operations suitably performed by a medical workflow assistance system including at least one microphone and an electronic processing device for derive actionable information from recorded audio and using that actionable information to modify or add to the medical workflow.

### DETAILED DESCRIPTION

As healthcare becomes more virtualized, additional information about the patient must come from untraditional sources. One approach is to use voice analytics, speech recognition, and voice biomarkers to uncover potentially new information about the patient. Neurological conditions, cardiovascular disease, brain injury, certain lung disorders, etc. can affect individual’s voice and speech patterns. AI-based voice analytics can play an important role in gaining a better understanding of a patient’s health status. For example, in a radiology setting, voice analytics can help streamline workflows, aid in diagnostics, and help ensure staff members and their mental health are tended to (i.e. capture early signs of burnout).

One of the drawbacks of virtualized healthcare is potentially missing out on patient cues due to limited face-to-face interactions. With expert technologists operating remotely and novice technologists or assistants responsible for patient interactions, information pertinent to a successful scan and/or diagnosis may be missed or misunderstood. Judgement of which piece of information imparted to the technologist by a patient is meaningful should not be left to be decided by inexperienced staff members. Analyzing audio recordings of patients, patient/staff interactions along the different stages of the workflow will 1) help streamline the workflow itself by identifying patient characteristics that could potentially delay or derail the exam, 2) provide verbatim patient history details that could be important for diagnosis, 3) play a role in identifying serious health conditions via voice biomarkers, and 4) help identify staff members experiencing undue stress and showing early signs of burnout.

The following relates to a system for managing communications between a patient undergoing a medical imaging examination; a local technologist who operates a medical imaging device to actually perform the imaging examination on the patient; and, in some embodiments, a remote expert who is on call to assist the local technologist. These parties may, in general, speak different languages, which can be a barrier to communication. In another scenario, the patient has some limited ability in the language as the local technologist (or remote expert), but the patient may lack sufficient skill in that language to communicate sufficiently to understand the imaging examination process and any actions the patient must take to ensure the examination goes as planned.

In a typical existing arrangement, an intercom is provided between the imaging bay and the control room. The intercom defaults to transmitting continuous audio from the imaging bay to the control room. If the technologist wants to talk to the patient, he or she presses an intercom button. Since the imaging bay is noisy for most modalities, pressing the intercom button simultaneously mutes the audio of the imaging bay in the control room. In a usual setup, the intercom uses a loudspeaker and microphone placed in the imaging bay, while in the control room either a loudspeaker and broad-area microphone or (more usually) a headset is used.

The disclosed communication system may optionally utilize an existing intercom, by feeding the audio from the imaging bay into a tablet or other electronic processing device located in the control room, where an application program (app) or other software running on the tablet sends the audio to a headset connected to the headphone jack of the tablet. The audio from the control room may be unmodified. However, if there is a language barrier between the patient and the imaging technician (for example, if they speak different languages, or if one actor has limited fluency in a common language) then the audio may be pre-processed by a signal processing chain including speech detection and then extracting any detected speech using speech-to-text or the like, machine translation from the patient’s language to the technologist’s language, and then text-to-speech to convert the translated text to audio that is played to the technologist via the headset. Additional or other audio processing may optionally be applied such as noise suppression algorithms to suppress known imaging device noise sources. To ensure the technologist does not miss possibly important noise features, the noise suppression may be applied only when patient’s speech is detected. If the technologist is also seeing video of the patient’s face, it is also contemplated to perform lip synching of the translated text. (If the audio processing takes a significant amount of time, e.g. a fraction of a second to a couple seconds or so, then the lip synching could additionally/alternatively involve delaying the video feed to synch with the translated audio output).

In the opposite direction, the technologist’s speech is similarly processed to provide any needed machine translation. However, in some disclosed embodiments, in this direction there is also a linguistic complexity adaptation step performed prior to the machine translation, in which any overly complex speech content (e.g. medical terms, overly long sentences, et cetera) are converted to simpler lay language. Additionally, the usual hardwired intercom button could be replaced by a softkey on the tablet and/or the tablet could automatically detect the technician’s speech and automatically mute the feed of audio from the imaging bay to the headset while the technologist is speaking.

To set up the disclosed system for a given imaging examination, the patient’s language is input to the system. In embodiments with linguistic complexity adaptation, a level of language proficiency may also be an input to the system.

In some embodiments, machine translation (without linguistic complexity adjustment) could also be applied for remote expert-local technician communication, which could facilitate expansion of the disclosed system to countries with many languages or in which the remote expert may be in a different country with a different language than the local technician. A communication link with a radiologist could be similarly enhanced.

The disclosed system can be also include a number of further variants, such as employing a video display to display the instructions using a graphical representation of a person communicating in American Sign Language (ASL) or another form of sign language if the patient is deaf and uses sign language, providing an audio splitter to enable the remote expert to speak directly to the patient, various automated dialog scripting options, and speech adjustments short of machine translation, such as suppressing a strong accent.

In some illustrative embodiments, the conventional intercom is not modified at all, and instead an audio splitter feeds the intercom audio to the tablet, and translated patient’s speech is displayed textually on the tablet screen. If the patient has a display as well, then the technician’s speech can be similarly displayed visually rather than being transmitted to the patient as audio speech. If the patient has no display, then the technologist can read the translated text. This latter option assumes the technologist has some familiarity with the patient’s language or at least understands the phonology of that language sufficiently to articulate the displayed translated text. For example, Spanish is a completely phonetic language, so the technologist with limited Spanish sufficient to know Spanish phonology could read the instructions after translation to Spanish, even if the technologist does not understand the meaning of the Spanish-language text.

In some illustrative embodiments, the disclosed apparatus analyzes recorded audio during patient/medical staff interactions to identify actionable information about the patient and/or staff member that is used to modify the clinical workflow. The disclosed systems and methods have broader applicability, especially (but not limited to) in the telemedicine sphere where information exchange efficiency is limited such that telemedicine interactions may especially benefit from the disclosed audio analysis techniques.

On the patient side, voice analysis can detect situations such as aggressiveness, anger, anxiety, indications of frailty, indications of mental impairment or disability, and so forth. Analysis of the speech can also be performed, i.e. natural language processing (NLP) of the speech content to extract relevant patient information. Finally, voice biomarkers of specific disease conditions may be detected. For example, analysis of breathing patterns of the patient (gasping, for example) may be detected and used to tentatively diagnose a respiratory disease such as emphysema or chronic obstructive pulmonary disease (COPD). This feedback can be actionable in modifying the clinical workflow to address the identified mental or physical conditional aspects of the patient, and/or can be recorded in the patient medical record for consideration by the patient’s physician. In the case of detected aggressiveness or anger, a call to hospital security could be issued. In some examples, patient distress could create a need for help, including connecting to the remote expert of the disclosed ROCC system. Additionally or alternatively, the detection of patient distress could automatically trigger other remedial action if the patient distress indicates an immediate and physical need. For example, detection of difficulty in the patient’s breathing could trigger an emergency call to medical staff indicating a patient in acute respiratory distress. Detection of situations mentioned above could also automatically trigger changes to the current workflow, additions to the current workflow and/or alerts/modifications to the scheduling. For example, detection of patient distress possibly indicative of an acute claustrophobic panic episode could trigger aborting an imaging scan and operating a robotic patient support to extract the patient from the imaging bore. The information can also be provided to the expert in case other issues arise, such that the expert can assess whether such additional information helps understand the issue with the examination. For example, if the image quality is insufficient, there can be analysis of the noise to determine breathing patterns, talking, or movement as the cause or contributing to the image quality issues. Thus, during a radiologist’s quality control review of an acquired medical image, detection of sounds indicative of patient motion during an imaging scan that acquired that image can be provided to the radiologist, for example as a note shown annotated to the image. If the radiologist concludes the image quality is unacceptable and also sees such an annotation, the radiologist is better positioned to advise the technologist on how to remedy the situation in a subsequent rescan.

On the staff member side, voice analysis can be used to detect anger, stress, impairment, an injury (for example, by directly detecting a sound indicating a possible injury such as a blunt-force strike sound, and/or by detecting a vocal response to an injury such as crying out in pain), or the like, and analysis of speech by NLP can be used to detect inappropriate language being used. This information can be used to determine if the staff member needs remedial training, or should be assigned time off, or if some other remedial action should be taken. In some examples, detection of impairment or injury can automatically trigger establishment of a communication connection with the remote expert. In addition, stress could be indicative that help is needed, thus creating a communication link to the remote expert of the disclosed ROCC system. Additionally or alternatively, detection of impairment or injury can trigger a call to hospital security. As another example, the nature of the examination, the discussion points, questions raised, etc. can also indicate where training is needed, whether it is technical, clinical, or related personal interactions. Information extracted by the NLP can also be used to automatically detect impending phase transitions in the workflow. For example, if the staff member says something like “We are all finished here” this can be a cue to automatically request patient transport such as a wheelchair.

To alleviate concerns that patient responses might be recorded (in the context of a partially automated telemedicine video call, for example), an initial welcome screen could be presented which states information derived from the vocal analysis, such as: “It sounds like you may be tired” so as to implicitly notify the patient that vocal analysis is occurring.

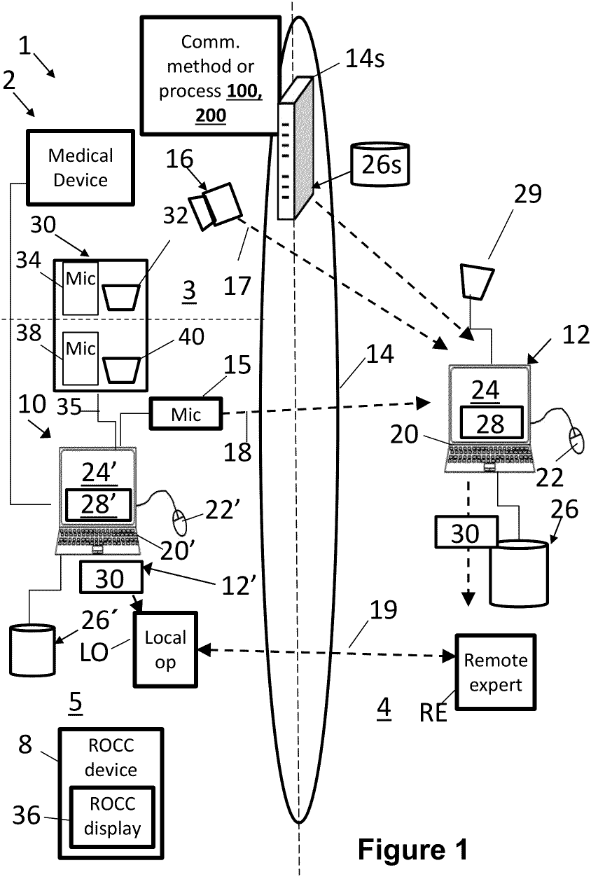

With reference to FIG. 1, an apparatus for providing assistance from a remote medical imaging expert RE (i.e., a radiologist) to a local radiologist technician or local technician operator LO is shown. Such a system is also referred to herein as a radiology operations command center (ROCC). While described in the illustrative context of an imaging examination performed in conjunction with an ROCC, the disclosed communication system embodiments for communicating between an imaging bay containing a medical imaging device and a control room containing a controller for controlling the medical imaging device are also suitably used in the absence of an ROCC, so as to provide improved communication between the patient and the imaging technologist in the control room. As shown in FIG. 1, a medical imaging device (also referred to as an image acquisition device, imaging device, and so forth) 2 is located in a medical imaging device bay 3, the remote expert RE is disposed in a remote service location or center 4, and the local operator LO operates a medical imaging device controller 10 in a control room 5. It should be noted that the remote expert RE may not necessarily directly operate the medical imaging device 2, but rather provides assistance to the local operator LO in the form of advice, guidance, instructions, or the like. Furthermore, in embodiments in which no ROCC is being used, the local operator LO simply corresponds to an imaging device operator or technologist (i.e., there is no remote expert in these embodiments).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities, and the discussion here focuses on a single imaging bay 3 and a single corresponding control room 5. Moreover, the remote service center 4 may provide service to multiple hospitals. The local operator LO controls the medical imaging device 2 via an imaging device controller 10 in the control room 5. The remote expert RE is stationed at a remote workstation or electronic processing device 12 (or, more generally, an electronic controller 12 or an electronic processing device 12).

The imaging device controller 10 includes an electronic processor 20′, at least one user input device such as a mouse 22′, a keyboard, and/or so forth, and a display device 24′. The imaging device controller 10 presents a device controller graphical user interface (GUI) 28′ on the display 24′ of the imaging device controller 10, via which the local operator LO accesses device controller GUI screens for entering the imaging examination information such as the name of the local operator LO, the name of the patient and other relevant patient information (e.g. gender, age, etc.) and for controlling the (typically robotic) patient support to load the patient into the bore or imaging examination region of the imaging device 2, selecting and configuring the imaging sequence(s) to be performed, acquiring preview scans to verify positioning of the patient, executing the selected and configured imaging sequences to acquire clinical images, display the acquired clinical images for review, and ultimately store the final clinical images to a Picture Archiving and Communication System (PACS) or other imaging examinations database. In addition, the remote service center 4 (and more particularly the remote workstation 12), and the control room 5 (in particular, the medical imaging device controller 5) are in communication with each other via a communication link 14, which typically comprises the Internet augmented by local area networks at the remote operator RE and local operator LO ends for electronic data communications.

As diagrammatically shown in FIG. 1, in some embodiments, a camera 16 (e.g., a video camera) is arranged to acquire a video stream 17 of a portion of the medical imaging device bay 3 that includes at least the area of the imaging device 2 where the local operator LO interacts with the patient, and optionally may further include the imaging device controller 10. The video stream 17 is sent to the remote workstation 12 via the communication link 14, e.g., as a streaming video feed received via a secure Internet link.

In other embodiments, the live video feed 17 of the display 24′ of the imaging device controller 10 is, in the illustrative embodiment, provided by a video cable splitter 15 (e.g., a DVI splitter, a HDMI splitter, and so forth). In other embodiments, the live video feed 17 may be provided by a video cable connecting an auxiliary video output (e.g. aux vid out) port of the imaging device controller 10 to the remote workstation 12 of the operated by the remote expert RE. Alternatively, a screen mirroring data stream 18 is generated by screen sharing software 13 running on the imaging device controller 10 which captures a real-time copy of the display 24′ of the imaging device controller 10, and this copy is sent from the imaging device controller 10 to the remote workstation 12. These are merely nonlimiting illustrative examples.

The communication link 14 also provides a natural language communication pathway 19 for verbal and/or textual communication between the local operator LO and the remote expert RE, in order to enable the latter to assist the former in performing the imaging examination. For example, the natural language communication link 19 may be a Voice-Over-Internet-Protocol (VOIP) telephonic connection, a videoconferencing service, an online video chat link, a computerized instant messaging service, or so forth. Alternatively, the natural language communication pathway 19 may be provided by a dedicated communication link that is separate from the communication link 14 providing the data communications 17, 18, e.g., the natural language communication pathway 19 may be provided via a landline telephone. In addition, the natural language communication pathway 19 can also be established between the remote expert RE in the remote service center 4, and the patient in the medical imaging device bay 3, thus allowing direct communication between the remote expert RE and the patient. These are again merely nonlimiting illustrative examples.

FIG. 1 also shows, in the remote service center 4 including the remote workstation 12, such as an electronic processing device, a workstation computer, or more generally a computer, which is operatively connected to receive and present the video 17 of the medical imaging device bay 3 from the camera 16 and to present the screen mirroring data stream 18 as a mirrored screen. Additionally or alternatively, the remote workstation 12 can be embodied as a server computer or a plurality of server computers, e.g., interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the workstation 12. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24.

The medical imaging device controller 10 in the control room 5 also includes similar components as the remote workstation 12 disposed in the remote service center 4. Except as otherwise indicated herein, features of the medical imaging device controller 10 disposed in the control room 5 similar to those of the remote workstation 12 disposed in the remote service center 4 have a common reference number followed by a “prime” symbol (e.g., processor 20′, display 24′, GUI 28′) as already described. In particular, the medical imaging device controller 10 is configured to display the imaging device controller GUI 28′ on a display device or controller display 24′ that presents information pertaining to the control of the medical imaging device 2 as already described, such as imaging acquisition monitoring information, presentation of acquired medical images, and so forth. The real-time copy of the display 24′ of the controller 10 provided by the video cable splitter 15 or the screen mirroring data stream 18 carries the content presented on the display device 24′ of the medical imaging device controller 10. The communication link 14 allows for screen sharing from the display device 24′ in the medical imaging device bay 3 to the display device 24 in the remote service center 4. The GUI 28′ includes one or more dialog screens, including, for example, an examination/scan selection dialog screen, a scan settings dialog screen, an acquisition monitoring dialog screen, among others. The GUI 28′ can be included in the video feed 17 or provided by the video cable splitter 15 or by the mirroring data stream 17′ and displayed on the remote workstation display 24 at the remote location 4.

FIG. 1 shows an illustrative local operator LO, and an illustrative remote expert RE. However, the ROCC optionally provides a staff of remote experts who are available to assist local operators LO at different hospitals, radiology labs, or the like. The ROCC may be housed in a single physical location or may be geographically distributed. The server computer 14 s is operatively connected with a one or more non-transitory storage media 26 s. The non-transitory storage media 26 s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14 s, various combinations thereof, or so forth. It is to be understood that any reference to a non-transitory medium or media 26 s herein is to be broadly construed as encompassing a single medium or multiple media of the same or different types. Likewise, the server computer 14 s may be embodied as a single electronic processor or as two or more electronic processors. The non-transitory storage media 26 s stores instructions executable by the server computer 14 s. In addition, the non-transitory computer readable medium 26 s (or another database) stores data related to a set of remote experts RE and/or a set of local operators LO. The remote expert data can include, for example, skill set data, work experience data, data related to ability to work on multi-vendor modalities, data related to experience with the local operator LO and so forth.

FIG. 1 also shows a communication system between the medical imaging device bay 3 and the control room 5. The communication system includes an intercom 30 which in the illustrative embodiment includes the following components. A bay audio speaker 32 and a bay microphone 34 are disposed in the imaging bay 3. Bay audio from the imaging bay 3 acquired by the bay microphone 34 is transmitted via a communication pathway 35 to a tablet computer 36 or the like in the control room 5 that is operatively connected with the communication pathway 35. The illustrative tablet computer 36 may, for example, comprise an iPad® available from Apple Corporation or an Android® tablet available from Samsung Corporation for example, but another type of electronic processing device such a notebook computer, or the controller of the imaging device itself, can also be used. The electronic processing device 36 is configured to (i) generate instructions to the bay audio speaker 32 for output by the bay audio speaker 32 and/or (ii) modify the bay audio and output the modified bay audio in the control room 5.

The intercom 30 also includes a control room microphone 38 disposed in the control room 5 and configured to receive instructions read by the local operator LO. The control room microphone 38 is connected with the bay audio speaker 32, and the bay audio speaker 32 outputs instructions from the local operator LO to the patient. In some examples, instructions read (or to be read) by the local operator LO are displayed on the display of the tablet computer or other electronic processing device 36, which may optionally also be a component of the ROCC device 8. The intercom 30 also includes a control room loudspeaker 40 configured to output speech from the local operator LO in the control room 5. It should be noted that in some embodiments the control room microphone 38 and control room audio speaker 40 may be embodied as a headset worn by the operator LO. It is also contemplated for the bay microphone 34 and bay audio speaker 32 to be embodied as a headset worn by the patient.

In addition, while not shown in FIG. 1, an additional intercom 30 can be established between the medical imaging device bay 3 and the remote service center 4, thereby allowing direct communication between the remote expert RE and the patient.

Furthermore, as disclosed herein the server 14s implements a communication method or process 100 of for communicating between the imaging bay 3 containing the medical imaging device 2 and a control room 5 containing the medical imaging device controller 10.

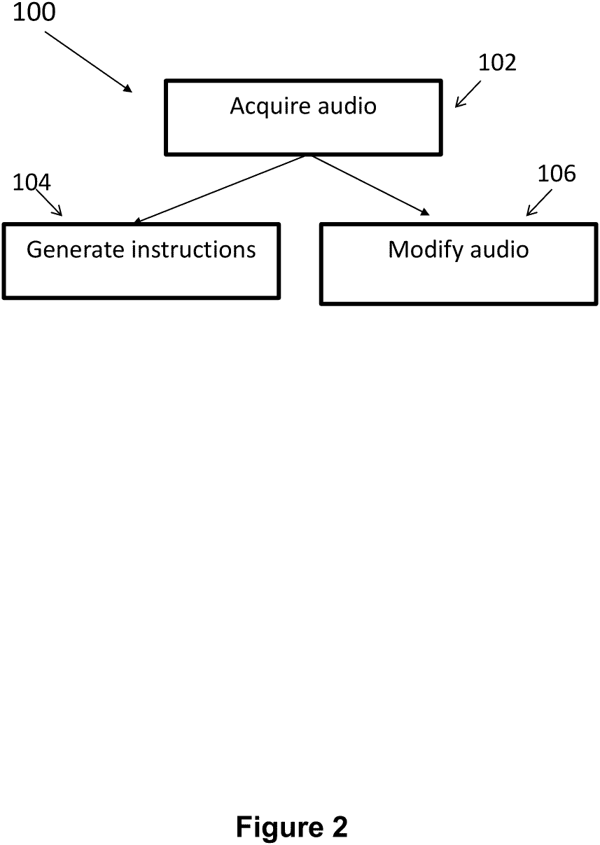

With reference to FIG. 2, and with continuing reference to FIG. 1, an illustrative embodiment of the method 100 is diagrammatically shown as a flowchart. At an operation 102, audio is received by the intercom 30. In one example, audio from the medical device imaging bay 3 (via the patient) is received by the bay microphone 34. In another example, audio from the control room 5 (via the local operator LO) is received by the control room microphone 38. The received audio is transmitted from the intercom 30 to the ROCC device 8.

At an operation 104, the ROCC device 8 is programmed to generate instructions. This can be performed in a variety of manners. In one example, the ROCC 8 is programmed to receive operator instructions from the local operator LO in a first language (i.e., a local operator language), and translate the operator instructions to generate the instructions in a second language that is different from the first language (i.e., a patient language). In another example, the ROCC 8 is programmed to receive operator instructions from the local operator LO, and perform natural language processing (NLP) on the operator instructions to reduce a linguistic complexity of the operator instructions to generate the instructions. In another example, the ROCC 8 is programmed to receive operator instructions from the local operator LO, and substitute at least one lay term or phrase for at least one medical term or phrase in the operator instructions to generate the instructions. In another example, the ROCC 8 is programmed to receive operator instructions from the local operator LO, and perform NLP on the operator instructions to modify a linguistic accent of the local operator LO to generate the instructions. In another example, the ROCC 8 is programmed to receive operator instructions from the local operator LO, and synthesize an audio signal representing the instructions which is transmitted from the control room 5 to the bay audio speaker 32 for output by the bay audio speaker 32. In another example, the control room microphone 38 is configured to receive instructions read by the local operator LO for output by the bay audio speaker 32 to the patient. The read instructions can be displayed on the display device 36 of the ROCC device 8. In another example, the display device 36 of the ROCC device 8 is visible to the patient in the medical imaging device bay 3, and the ROCC device 8 is programmed to generate a sign language representation of the instructions that is displayed on the display device 36 for visualization by the patient. These are merely examples and should not be construed as limiting.

At an operation 106 (which is not mutually exclusive with the operation 104), the ROCC device 8 is programmed to modify the bay audio and output the modified bay audio in the control room 5. In one example, the ROCC device 8 is programmed to extract speech in a first language from the bay audio (i.e., the patient language), and translate the extracted speech to a second language different from the first language (i.e., the local operator language) to generate the modified bay audio comprising the extracted speech translated to the second language. The extracted speech translated to the second language can be displayed on the display device 36 of the ROCC device 8. In another example, the ROCC device 8 is programmed to modify the bay audio by performing noise suppression on the bay audio. In another example, the ROCC device 8 is programmed to output the modified bay audio in the control room 5 as audio played by the control room audio speaker 40. These are merely examples and should not be construed as limiting.

In some embodiments, the natural language pathway 19 between the local operator LO and the remote excerpt RE can comprise a remote assistance communication path. The ROCC device 8 is programmed to translate first speech or text generated by the operator in a first language (i.e., the local operator language) to a second language (i.e., a remote expert language) and transmit the first speech or text translated to the second language to the remote expert via the remote assistance communication path to the remote workstation 12, and translate second speech or text generated by the remote expert in the second language to the first language and transmit the second speech or text translated to the first language to the operator via the remote assistance communication path to the ROCC device 8. Moreover, the natural language pathway 19 can be established between the remote excerpt RE and the patient. The remote workstation 12 (instead of the ROCC device 8) can perform the operations 102-106 to modify audio communications via the intercom 30 between the remote expert RE and the patient.

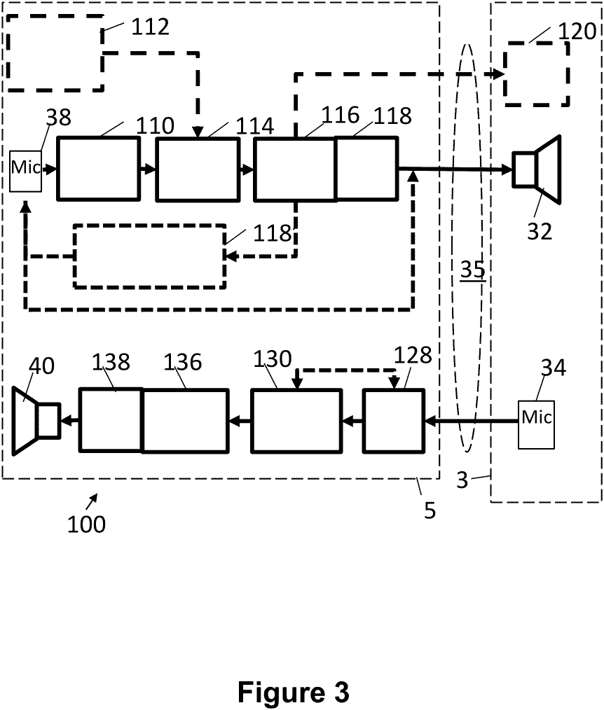

With reference to FIG. 3, a further embodiment of the communication system is described. In FIG. 3, operations performed in the control room 5 are delineated by a left-hand box and operations performed in the imaging bay 3 are delineated by a right-hand box. The top portion of FIG. 3 depicts communication from the operator LO in the control room 5 to the patient in the imaging bay 3, while the lower portion of FIG. 3 depicts communication from the patient in the imaging bay 3 to the operator LO in the control room 5. FIG. 3 depicts a non-ROCC embodiment, and hence the only actors are the operator LO and the patient.

With reference to the top portion, an instruction for the patient is produced the operator LO speaking into the microphone 38 with the speech extracted in an operation 110 (for example, by transcription software performing speech-to-text conversion), or the instruction is automatically produced by the imaging device controller in an operation 112, e.g. issuing a standard preprogrammed instruction. The instruction is assumed to be in a first language which is the language of the operator LO, or the language assumed by the imaging device controller. The resulting instruction is processed by an optional linguistic complexity reduction process 114 which, for example, may replace any technical terms with terms more likely to be understood by a layperson. The linguistic complexity reduction process 114 may also perform other complexity reduction, such as breaking a long sentence into a shorter sentence, and/or translating the instruction to an expression in the first language using a reduced vocabulary (e.g., a vocabulary in the first language with only 1000 of the most commonly used words in the first language). In a translation operation 116, the instruction is translated from the first natural language to a second natural language which is the language preferred by the patient. For example, if the operator speaks English and the patient speaks Spanish, then the translation operation 116 translates the (optionally complexity-reduced) instruction from English to Spanish. In a speech synthesis operation 118, the instruction now translated into the second language of the patient is converted to audio by speech synthesis, and the synthesized spoken instruction in the second language is conveyed via the communication path 35 to the imaging bay where it is played by the bay audio speaker 32. In a variant embodiment, if the operator has limited command of the second language but is competent with the phonology of the second language (and thus the operator can articulate the language), then in an operation 118′ the translated speech in the second language output by the translator 116 is displayed on the display of the tablet 36 so that the operator can then read the instruction in the second language to the patient using the control room microphone 38. In a variant embodiment of this latter path, if the operator articulates the second language with a strong accent, then audio signal processing can be performed to reduce this strong accent (operation not shown in FIG. 3). In yet another variant, if the patient is deaf then the second language may be American Sign Language (ASL) or another sign language, and the translated speech may be displayed in sign language using an ASL display 120 disposed in the imaging bay and visible to the patient.

With reference now to the bottom portion of FIG. 3, the communication path from the patient in the imaging bay 3 to the operator in the control room 5 is diagrammatically shown. In an optional operation 128, noise suppression may be performed on the audio received from the bay microphone 34 via the communication path 35, followed by speech extraction 130 analogous to the speech extraction 110. The optional noise suppression 128 suppresses noise generated by the imaging device 2 or other noise sources in the imaging bay 3 such as air handling systems. However, since it may be important for the operator in the control room to hear noise generated by the imaging device 2 that might be indicative of a problem with the imaging device 2, in some embodiments the noise suppression 128 and the speech extraction 130 are linked so that, for example, the noise suppression 128 is applied only when the speech extraction 130 is not detecting any speech to extract. Additional or other approaches can be used, such as using frequency-selective noise filtering in the operation 128 to avoid suppression of certain types of noise that are likely to be diagnostic of a problem with the imaging device 2.

In an operation 132, speech extracted by the speech extraction 130 is expected to be in the second language, that is, the language preferred by the patient, whereas the operator prefers the first language. Accordingly, a translation operation 136 is performed to translate the patient’s speech from the second language to the first language, followed by speech synthesis 138 to convert the translated speech in the first language to audio which is played by the control room audio speaker 40. Additionally or alternatively, the translated speech in the first language can be displayed on the display of the tablet computer 36.

Although not shown in FIG. 3, the communication system may include other features. For example, the communication system may provide a muting feature to mute the output of the control room speaker 40 when the operator is issuing instructions to the patient, so as to avoid a feedback loop where sound from the imaging bay is amplified. In embodiments employing the operation 118′, the initial instruction spoken by the operator in the first language is not conveyed to the patient via the communication path 35; rather, the communication path 35 is only switched to communicate the instruction in the second language read from the display produced by the operation 118′.

The communication system of FIG. 3 provides communication between the control room 5 and the imaging bay 3, to enable the imaging technologist to communicate with the patient. Advantageously, this communication system provides support for communication when the imaging technologist and patient speak different languages, and/or when the patient has limited comprehension of complex medical linguistics used by the imaging technician, and/or provides for suppression of noise when transmitting audio from the imaging bay 3 to the control room 5.

In an ROCC context, an analogous communication system to that of FIG. 3 can provide direct communication between the remote expert RE and the patient in the imaging bay 3. To do so, the control room microphone 38 and control room audio speaker 40 of the communication system of FIG. 3 can be replaced by a microphone and speaker of (or operatively connected with) the remote workstation 12 operated by the remote expert RE. In this variant, the communication system provides support for communication when the remote expert RE and patient speak different languages, and/or when the patient has limited comprehension of complex medical linguistics used by the remote expert RE, and/or provides for suppression of noise when transmitting audio from the imaging bay 3 to the remote workstation 12.

In a further variant that can be employed in the ROCC context, an analogous communication system to that of FIG. 3 can provide communication between the remote expert RE and the imaging technologist in the control room 5. To do so, the control room microphone 38 and control room audio speaker 40 of the communication system of FIG. 3 can be replaced by a microphone and speaker of (or operatively connected with) the remote workstation 12 operated by the remote expert RE; and the imaging bay microphone 34 and imaging bay audio speaker 32 of the communication system of FIG. 3 can be replaced by the control room microphone 38 and control room audio speaker 40. In this variant, the communication system provides support for communication when the remote expert RE and imaging technologist speak different languages, and/or when the imaging technologist has lesser knowledge of complex medical linguistics than the remote expert RE (for example, if the imaging technician is a new hire with limited training and experience).

It will be still further appreciated that in the ROCC contexts two or all three of these communication systems can be provided, that is: a communication system between the control room 5 and imaging bay 3 (as shown in FIG. 3); a communication system between the remote expert workstation 12 and imaging bay 3 (first variant); and/or a communication system between the remote expert workstation 12 and the control room 5 (second variant). Such a combination of communication systems can be particularly useful in an ROCC system that spans multiple regions or countries such that, for example, the remote expert may be located in a different country than the control room and imaging bay. This can enable, for example, a remote expert located in a developed country to provide assistance for an imaging session performed in a developing country.

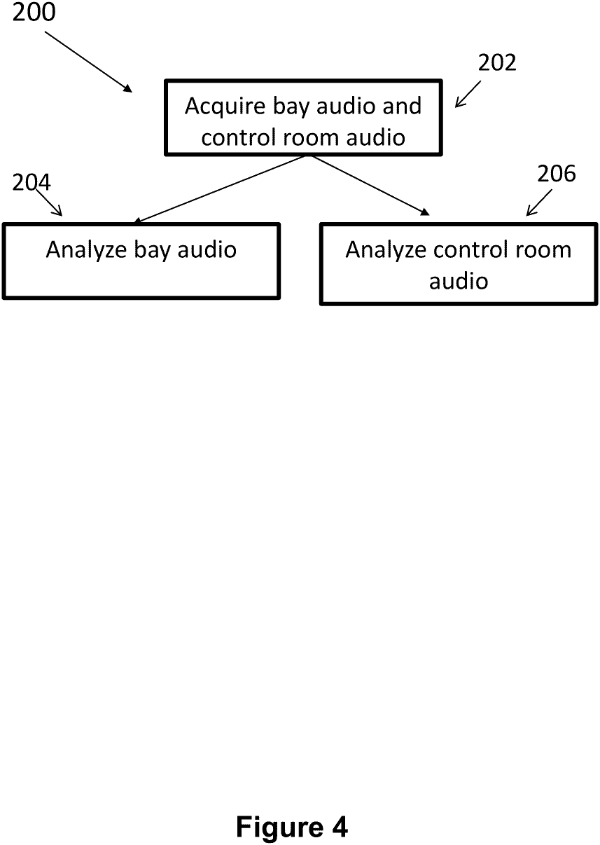

FIG. 4 shows an alternative embodiment of the method 200 diagrammatically shown as a flowchart. At an operation 202, audio is received by the intercom 30. In one example, audio from the medical device imaging bay 3 (via the patient) is received by the bay microphone 34. In another example, audio from the control room 5 (via the local operator LO) is received by the control room microphone 38. The received audio is transmitted from the intercom 30 to the ROCC device 8.

At an operation 204, the ROCC device 8 is programmed to analyze the bay audio acquired by the bay microphone 34. This can be performed in a variety of manners. In one example, the ROCC device 8 is programmed to analyze the bay audio to detect a mental or physical condition of the patient from the bay audio. A workflow of an imaging examination can be modified based on the detected mental or physical condition of the patient. In another example, the ROCC device 8 is programmed to perform an NLP process on the bay audio to extract patient information of the patient. The workflow can also be modified based on the extracted patient information. In a further example, the ROCC device 8 is programmed to detect one or more voice biomarkers of the patient (i.e., does the patient sound stressed, or is in trouble), and the workflow can also be modified based on the voice biomarker(s) of the patient. In each of these examples, the ROCC device 8 can generate and output an alert based on the analyzed bay audio. These are merely examples and should not be construed as limiting. In addition, the display device 36 of the ROCC device 8 can display a message generated by the ROCC device 8 for visualization by the patient in the imaging bay 3. The message can indicate to the patient that a vocal analysis process is being performed on the bay audio.

In some embodiments, the bay audio is transmitted to the remote service center 4 via a second intercom 30 disposed in the remote service center 4. The remote electronic processing device 12 is configured to analyze the bay audio.

At an operation 206 (which is not mutually exclusive with the operation 204), the control room microphone 38 is configured to acquire control room audio in the control room 5. The ROCC device(8 is programmed to analyze the control room audio acquired by the control room microphone 38. In one example, the ROCC device 8 is programmed to analyze the bay audio to detect a mental or physical condition of the local operator LO from the control room audio. A remedial action of the local operator LO can be determined based on the detected mental or physical condition of the local operator LO (i.e., whether the local operator LO needs additional training, a refreshment training session, repudiation from a superior, and so forth).

In another example, the remote electronic processing device 12 is programmed to perform an NLP process on the bay audio or control room audio to determine a phase transition in an imaging workflow of an imaging examination. The workflow of the imaging examination can be modified based on the extracted patient information.

The foregoing example is in the context of the ROCC of FIG. 1. However, the approach of analyzing audio acquired during an interaction between a patient whose is the subject of a medical workflow and a medical professional interacting with the patient during the medical workflow to derive actionable information that is used to modify or add to the medical workflow can be applied to a wide range of medical workflows, such as medical imaging examination workflows (with or without the ROCC), telehealth workflows, in-person medical encounters in which the audio is recorded, or so forth. As used herein, “telehealth” is to be understood as encompassing systems and methodologies for providing medical care to a patient from a remote location via a telephonic or video call or the like. In the art, telehealth may be referred to by similar nomenclatures such as telemedicine, remote healthcare, virtual healthcare, or so forth, all of which are intended to be encompassed by the term “telehealth” used herein.

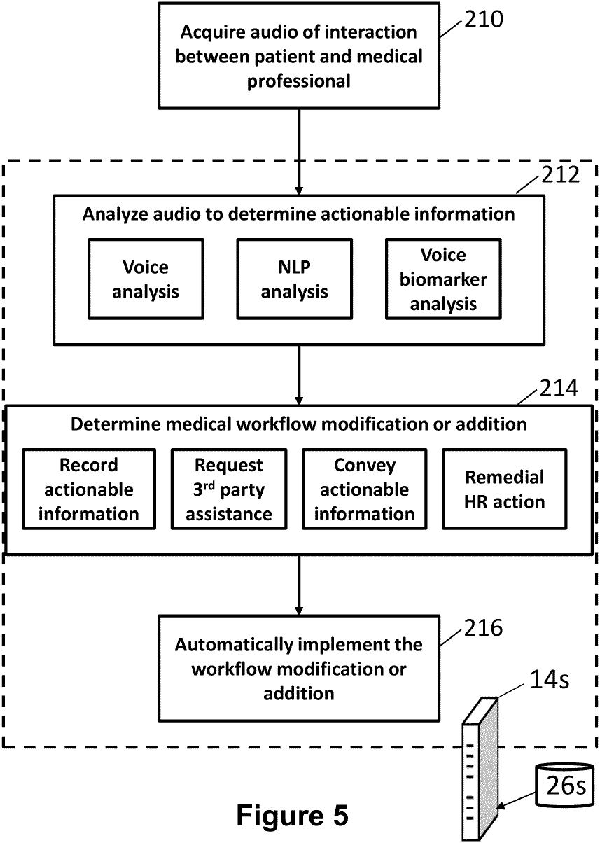

With reference to FIG. 5, a method is diagrammatically shown, which is suitably performed by a medical workflow assistance system including at least one microphone that acquires audio of an encounter of a patient with a medical professional during an episode or stage of a medical workflow, and an electronic processing device programmed to derive actionable information from the acquired audio and to use that actionable information to modify or add to the medical workflow. By way of nonlimiting illustrative example, the at least one microphone can be: one or both microphones 34, 38 of the ROCC previously described in the case of the episode or stage of the medical workflow being a medical imaging examination; a microphone disposed in a room within which an in-person interaction between a patient and a medical professional occurs; the microphones of telephone or smartphone handsets used in a telephonic telehealth session conducted between the patient and a medical professional; the microphones of video call telehealth session conducted between the patient and a medical professional; and/or so forth. As diagrammatically shown in FIG. 5, the method includes an operation 210 in which audio of the interaction between the patient and the medical professional is acquired using the at least one microphone. Subsequent operations 212, 214, and 216 are suitably automated, for example performed by the server computer 14s by reading and executing instructions stored on the non-transitory storage medium or media 26s.

In an operation 212, the electronic processing device 14s analyzes the audio acquired in the operation 210 by the at least one microphone to determine actionable information about the patient and/or the medical professional. Initially, this operation 212 entails disambiguating the voice of the patient and the medical professional (or medical professionals; while this example refers to a single medical professional it is to be understood the “medical professional” may include one, two, or more medical professionals). The medical professional can be a doctor, nurse, hospital or laboratory receptionist, physical therapist, imaging technician, or other person involved in the medical workflow. The patient may be an in-patient (i.e. admitted to the hospital) or an out-patient (who, in the case of a telehealth workflow may never actually set foot inside the hospital). To disambiguate (i.e. separate out) the patient’s voice and the health professional’s voice, various approaches can be used. In general, the voice analysis can readily distinguish two voices by average sound volume, average pitch, average speed, and/or so forth. To determine which voice is assigned to the patient and which is assigned to the medical professional, approaches such as detecting keywords using natural language processing (NLP) analysis of the voices can be used, e.g. NLP extraction of the phrase “I am doctor Jones” can be used to identify the speaker as a health professional (and more particularly a doctor). If some standard phrasing is used in medical encounters, such as asking the patient to provide his or her birthdate as a patient identification verification check, then detection of a spoken date early in the encounter can be used to identify the patient. In another approach, medical professionals on staff or authorized to work at the hospital or other medical institution can have their voices prerecorded to establish voice signatures of all medical professionals, and thereafter the medical professional can be identified by matching his or her voice signature and anyone not matched to a voice signature can be identified as a patient. In the case of telehealth encounters, a priori assignment of identities by the video call software (for example) can be used to readily assign voices to patient and medical professional, and a similar assignment can be done in the ROCC context based on which microphone 34 or 38 is recording the voice. These are merely nonlimiting illustrative examples.

With the voices of the respective patient and medical professional identified, the operation 212 can then proceed with analyzing the audio acquired in the operation 210 by the at least one microphone 34, 38 to determine actionable information about the patient and/or the medical professional. For example, with respect to the patient, voice analysis of the patient can be used to identify signs of aggression, annoyance, anger, anxiety, frailty, impairment, cognitive limitation, and/or so forth. Speech recognition (i.e., NLP) can be used to identify potentially important information (i.e., saved in notes for a radiologist) based on a type of examination the patient is receiving, adding notes to a PACS if the patient complains of pains, extravasation, etc. during the examination, a patient’s language proficiency and communication ability, and so forth. Analyzing biomarkers of the patient can be used to detect lung problems (i.e., a patient may have difficulty following breath hold protocols), brain injuries, cardiac diseases or other conditions that may require follow-up procedures or may show up as indicational findings, neurological impairment (i.e., a patient may have difficulty following instructions, and so forth). As one illustrative example, a respiratory condition of the patient may be detected by analyzing breathing of the patient in the audio acquired by the at least one microphone. As another example, frailty of the patient may be detected based on one or more of articulation rate, mean fundamental frequency, and/or voice intensity range of speech of the patient in the audio acquired by the at least one microphone. As another example, cognitive limitation may be detected by the medical professional frequently repeating questions before the patient provides an answer. As yet another example, a medical condition of the patient may be identified based on words or phrases obtained by the NLP of speech of the patient in the audio. These are merely nonlimiting illustrative examples.

Likewise, the operation 212 analyzes the audio of the voice of the health professional to determine actionable information about the medical professional. For example, voice analysis of the medical professional’s voice may be applied to detect signs of tiredness, anger, depression, or so forth, possibly indicative of burnout, disinterest and boredom which may indicate a need for greater challenges, diversity of cases, opportunities for growth and development, signs of continuous or constant stress may indicate a need for additional training, and so forth. In another example, speech recognition (i.e., NLP) can be used to identify issues such as detecting abusive, inappropriate language, abrasive behaviors towards patients or fellow staff members, recognize voice commands to trigger calls to other patients (i.e., a radiology, remote expert, or so forth), initiate emergency protocols when help is needed (call 911, alert security, and so forth), recognize workflow stages based on words or phrases detected (e.g., “last scan,” “five minutes left in the examination, etc.), create timelines based on recognition of workflow activities, and so forth. As one illustrative example, voice analytics may be performed on speech of the medical professional in the audio to detect stress or exhaustion of the medical professional. In another example, the voice analytics may detect slurred speech of the medical professional indicative of impairment by alcohol or drugs. Again, these are merely nonlimiting illustrative examples.

In an operation 214, a modification of or addition to the medical workflow is determined based on the actionable information extracted in the operation 212. By way of nonlimiting illustrative example, for the patient such a modification or addition might include one or more of: recording an indication of a detected medical or physical condition of the patient in an electronic patient record of the patient; requesting assistance of a third party selected by the electronic processing device based on the detected medical or physical condition of the patient; communicating the indication of a detected medical or physical condition of the patient to the medical professional involved in the interaction; and/or communicating the indication of the detected medical or physical condition of the patient to a medical doctor treating the patient. In the example of requesting assistance of a third party, that third party could be selected as a security officer if the detected medical or physical condition is likely to be associated with the patient becoming physically aggressive (e.g., detected aggression or anger), or could be selected as a medical doctor (e.g., if an acute medical condition is detected and the medical professional involved in the interaction is a nurse, receptionist, or other medical professional who is not a medical doctor), or could be a transport assistant (e.g., if the detected condition is frailty of the patient such that he or she may need a wheelchair or other transport assistance), or so forth. In workflow modifications or additions relating to communicating an indication of a detected medical or physical condition, this could be communicated privately to the medical professional involved in the interaction (e.g., via a headset worn by the medical professional) or could be communicated to the patient’s doctor via a constructed email, text message, or the like. These again are nonlimiting illustrative examples.

In the case of actionable information about the medical professional involved in the interaction, some examples of possible medical workflow modifications or additions include: recording an indication of a detected medical or physical condition of the medical professional in an electronic employee record of the medical professional; requesting assistance of a third party selected by the electronic processing device based on the detected medical or physical condition of the medical professional; or performing a remedial human resources (HR) action such as suspending the medical professional or scheduling remedial training for the medical professional. (In the case of a suspension, the suspension would likely need to be affirmed by HR personnel before taking permanent effect). In the example of requesting assistance of a third party, that third party could be selected as a security officer if the detected medical or physical condition is likely to be associated with the medical professional becoming physically aggressive, or if the medical condition is inebriation or drug impairment detected by slurred speech such that the medical professional should not be providing patient services at this time. The remedial training could be, for example, sensitivity training if the actionable information is the medical professional being abusive to the patient, for example.

In an operation 216, the workflow modification or addition is automatically implemented by the electronic processing device 14s. For example, information can be actually added to the electronic patient record of the patient, and/or to the electronic employee record of the medical professional, if such information recordation is the determined medical workflow modification or addition. In the case of adding information, this would typically be done in a tentative manner, e.g. added with a notation that the added medical information must be confirmed by the patient’s doctor, or that information added to the electronic employee record of the medical professional must be affirmed by HR personnel. In the case of the workflow modification or addition entailing requesting assistance of a third party, the electronic processing device 14s can implement this directly by issuing an alert to said third party, preferably including an indication of the actionable information determined in operation 212 that led to issuing the alert. Similarly, workflow modification or addition involving conveying information to a medical doctor, and/or to the medical professional involved in the encounter with the patient, this can comprise an automatically constructed and sent text message, synthesized speech message, or so forth presenting the actionable information to the recipient. Again, these are merely nonlimiting illustrative examples.

In the following, some further examples are provided.

### EXAMPLE

AI technologies/conversational AI can be used in imaging workflow to translate communication between the local operator LO and the patient and for standard workflow guidance and patient self-positioning. The remote expert RE can guide the patient directly by looking at the cameras and via voice communication. NLP models such as BERT or various machine learning/deep learning models can be used as AI assistants between the tech and the patient. An audio splitter 15 can be used in conjunction with the ROCC device 8 to allow communication between remote expert RE, the local operator LO, and the patient simultaneously. The ROCC device 8 can be connected to the bay audio speaker 32 via audio cables, Bluetooth or wireless technology easily. The bay microphone 34 can also pick up communication from the patient, translate if necessary and relay it back to local or remote technologist via the ROCC device 8. Additional embodiments include a vocabulary assistant for the local operator LO that speak the patient’s language partially but lack the necessary vocabulary to be able to guide the patient, accent adjustment for the patients/local operator LO with a heavier accent, lip synching of the video during translation, auto-modification of technologists’ voice to the translated language to allow for tech to communicate with their own voice in a different language, auto captioning during video communication for patients with auditory deficiencies and language simplification to allow patient to understand the techs and radiologists that use medical jargon that is not familiar to the patient more frequently.

Referring back to FIG. 1, the ROCC device 8 includes a translator module 42 configured to translate speech/communication between the local operator LO/remote expert RE and the patient using the AI algorithms trained specifically for translation tasks. The ROCC device 8 also includes a guidance module 44 configured to store basic instructions to guide the patient through the radiology imaging workflow, such as instructing the patient to lie down in a specific position, to hold their breath, to squeeze the ball in the case of a discomfort, etc. and is capable of understanding basic/frequently asked questions from the patient and answering them in a friendly manner. NLP algorithms specifically trained for the purposes of instructing/answering questions from the patient can be implemented, and can aid in translating the communication if the patient speaks in a certain language.

An AI component 46 includes AI algorithms such as Machine Learning/Deep Learning/Transformer models required for tasks such as speech recognition, text-to-speech, NLP, and translation to facilitate the communication between local operator LO/remote expert RE and the patient and allow the patient to self-guide through the radiology imaging workflow when the local operator LO is busy.

Before the imaging procedure, the patient’s communication requirements are assessed. This could be done already at the reception desk, via a self-check-in app, via a questionnaire, or other means. The patient’s communication requirements may include, but are not limited to, a preferred language, a second language, a level of proficiency of preferred language, a level of proficiency of second language, a level of knowledge of medical terms, patient age, special technical requirements (such as a hearing aid), and so forth. Languages may even include sign languages, which requires a visual instead of an audio transcription technology. The patient’s communication requirements determine the settings of the translator module 42.

The translator module 42 is configured for translating the local operator’s spoken language to the patient’s preferred language. If the ROCC system is not capable of providing the patient’s preferred language, the patient’s second language is chosen instead. (This could even be extended to further languages of that patient’s choice.)

As an intermediate step between the interpretation of the local operator’s input and the translated output, the complexity level of the language is adapted to the patient’s communication requirements: The adaptation step includes exchanging one word for another (e.g., exchange a medical term for a lay language term), changing the complexity of phrasing (e.g., splitting a long sentence into two or more shorter ones), or exchanging one multi-word expression for another (e.g., exchange a medical expression for a lay language expression), depending on the patient’s communication requirements. It is possible that input and output language are the same and only the complexity level is adapted. In this case, if the complexity of the input sentence is found to match the complexity requirements of the patients, the spoken sentence may be passed on to the patient without changes. The communication system is further configured to translate the patient’s spoken language to the technologists spoken language without change of language complexity.

In some embodiments disclosed herein, instead of processing the language directly, the disclosed system can even provide the local operator LO with proposed expressions or sentences in the patient’s language that are suitable to be used in the current situation, adapted to the patient’s language complexity requirements. This may enable the local operator LO who has some but limited knowledge of the patient’s language to still communicate with the patient directly by applying the expressions proposed by the disclosed system.

The disclosed system may also include a number of pre-defined sentences or expressions that can be triggered manually or automatically in a given situation. Automatic triggering of spoken language can be extended to other vendor’s equipment by being integrated into the ROCC platform and triggered based on the examination state known to the ROCC system.

A manual triggering can be implemented by providing the local operator LO with a number of choices of fixed sentences to select from, which will then be output in the patient’s language, adapted to the required complexity level.

In another embodiment, the disclosed system also provides sign language support by visualizing an animated avatar on a screen facing the patient. This translation option may only work in one direction, from the local operator LO to the patient.

In another embodiment, the disclosed system is used for the local operator LO, where the text messages or voice memo sent could automatically be translated into the preferred languages. The system could also be used for converting the voice memo to text during the chat and vice versa.

In another embodiment, several features that utilize voice analytics along the workflow stages in a hospital setting such as a radiology department can be implemented. Recording opportunities for such voice analytics can include a patient being contacted over the phone to schedule an appointment, a screening call to ensure patient understands instructions, review safety questions, patient intake at the reception, patient wait in the lobby, patient education with technologist, patient in the dressing / changing room, patient conversation with the nurse (if IV placement is required), patient comments while in the scanner, patient remarks following the scan, and so forth. Audio recordings can be used to help patients (deliver better patient care) and staff - whether local or virtual (detect early signs of burnout in staff members).

Voice analytics can be determined to better meet patient needs. During the patient journey (whether real-life or virtual), patient voice analytics (analysis of audio recordings of patient interactions) may provide valuable insights into patient’s state of mind or sentiments. Multiple peer reviewed articles have been published about verbal aggression detection. Likelihood of verbal aggression can be teased out by applying signal processing techniques on acoustic cues. Identifying aggressive or angry patients early on can help staff members take precautions, pre-emptive actions and diffuse the situation. Similarly, promising studies have shown correlations of acoustic measures such as articulation rate, mean fundamental frequency, intensity range, etc. with frailty. Identifying frail individuals in advance can help staff members make appropriate arrangements to meet patient needs without derailing the operations of the entire department. In short, established acoustic measures could be used to identify some common patient states of interest (anger, frailty, anxiety, miscomprehension, tiredness, disorientation, stress). Identifying these patient states could help staff members make the necessary scheduling changes, arrange for additional resources, etc. With additional data, voice analytics models can be further refined and customized. Matching the acquired audio data with patient behavior can help train setting-specific, nuanced models. By early identification of patients who need additional time, resource, modified (e.g. shorter, quieter, and so forth) diagnostic procedures and attention, patient care can be improved, while also guarding the workflow against some common disruptions.

With hospitals existing is silos and patients often receiving care from different medical professionals distributed all over the map, patient medical history can be patchy or entirely missing. Unfortunately, diagnostics requires context. Radiologists, for instance, may put the reading of a study on hold if patient’s history is incomplete. In a radiology setting, patients often relay pertinent information to nurses or technologists. That information rarely makes it to the radiologists, whose job it is to combine what is known and interpret images within appropriate patient context. In short, there is a lost opportunity. By recording patient communications during one, but also over the course of possibly many different procedures, and using speech recognition, patient information that could otherwise be lost can be gathered. To help a medical professional (such as a radiologist) make sense of the data: 1) an AI model can be trained using input from doctors and finalized reports to recognize information that may be pertinent to diagnosis (could be exam specific). A module can also be provided to review the available EMR data and supplements the missing pieces of Information and/or prompts the staff to confirm the information with the patient before updating EMR with new patient details. In addition, speech recognition can be used to keep track of events during the exam itself - from patient complaints to adverse reactions, etc., taking the burden from the technologist in having to record these events manually while taking care of patients. A speech detection model can be trained to identify certain common phrases and be customized for specific settings.

Vocal patterns could be used as disease biomarkers. One approach is to transfer an audio recording into a visual representation and train AI models to match voice patterns to diseases. In the absence of reliable patient history, such auditory biomarkers could guide and support the diagnostic process. In the right context, within the right timeframe, such additional information could help interpret incidental findings, help establish appropriate follow-up, etc. Additionally, voice biomarkers may help in identifying patients likely to struggle following instructions (respiratory or cognitive problems) and could pave the way for more customizable slot allocation during scheduling. In addition, they could serve privacy respecting goals, i.e. by not storing the spoken words explicitly.

In addition to patient analytics, audio of a staff member can be collected ana analyzed. Burnout is a psychological condition that typically emerges when an individual undergoes prolonged periods of work-related stress. While highly variable, some of the features of burnout include emotional exhaustion, apathy towards patients or colleagues, overwhelming sense of inadequacy and failure. Burnout amongst medical professionals is common and can lead to unfortunate consequences not only for the individual himself but for his patients as well. Identifying instances of staff burnout or identifying early signs or signs of impeding burnout can create opportunities for intervention, help, and encourage better health and well-being in the workplace. Acoustic measures and spectrograms from audio recordings, combined with questionnaires evaluating staff mental health and stress levels in a longitudinal fashion could be used to train AI models to identify instances of burnout, mental health struggles, etc.

There are situations where the local inexperienced technologist can encounter critical/distress situations while scanning the patient. In such scenarios the local technologist can use voice commands to activate emergency protocols or call a radiologist or an expert user to get help while the local technologist rushes to help the patient in the scanner room.

In certain instances (especially in remote setting), there might be a need to use speech recognition to identify workflow stage in progress or to map out progression of the exam. By recognizing specific phrases, a timeline of activities/events that have occurred so far can be created. This could help orient a remote expert summoned to help a local tech and could be used for QA/QC purposes.

In one embodiment, the patient’s communication ability, such as language proficiency and capability of understanding and answering questions, is analyzed by a speech recognition algorithm in any communication of the patient with hospital staff. Based on this analysis, the expected communication difficulties in upcoming examinations can be estimated. For example, as is indicated by clinical studies, the ability of a patient to understand and follow communication with the technologist has an impact on the MR examination workflow, such as duration of workflow steps or likelihood of scan repeats. The communication ability can therefore be used to estimate the required slot time and potential expert support requirements.

In one embodiment, all speech and voice processing takes place in a processing unit directly attached to the audio acquisition device. For example, a processing unit could be integrated with a microphone and positioned in an examination room. The audio signal is thus processed at the source and only the extracted features are transmitted to other IT systems. In this way, privacy can be preserved because the original audio signal is not accessible and is not stored anywhere.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

In the foregoing detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials, and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

1. A communication system for communicating between an imaging bay containing a medical imaging device and a control room containing a controller for controlling the medical imaging device, the communication system comprising:
an intercom including:
a bay audio speaker disposed in the imaging bay;
a bay microphone disposed in the imaging bay; and
a communication path via which bay audio from the imaging bay acquired by the bay microphone is transmitted to the control room and via which instructions are transmitted from the control room to the bay audio speaker for output by the bay audio speaker; and
an electronic processing device operatively connected with the communication path and programmed to at least one of:
(i) generate the instructions; and/or
(ii) modify the bay audio and output the modified bay audio in the control room.

2. The communication system of claim 1, wherein the electronic processing device is programmed to generate the instructions by operations including:
receiving operator instructions from an operator in a first language; and
translating the operator instructions to generate the instructions in a second language that is different from the first language.

3. The communication system of claim 1, wherein the electronic processing device is programmed to generate the instructions by operations including:
receiving operator instructions from an operator; and
generating the instructions by performing natural language processing on the operator instructions to reduce a linguistic complexity of the operator instructions.

4. The communication system of claim 1, wherein the electronic processing device is programmed to generate the instructions by operations including:
receiving operator instructions from an operator; and
generating the instructions by substituting at least one lay term or phrase for at least one medical term or phrase in the operator instructions.

5. The communication system of claim 1 wherein the electronic processing device is programmed to generate the instructions by operations including:
receiving operator instructions from an operator; and
generating the instructions by performing natural language processing on the operator instructions to modify a linguistic accent of the operator.

6. The communication system of claim 1, wherein the electronic processing device is programmed to:
generate the instructions; and
synthesize an audio signal representing the instructions which is transmitted from the control room to the bay audio speaker for output by the bay audio speaker.

7. The communication system of claim 1, further comprising:
a microphone disposed in the control room to receive the instructions read by an operator and connected with the communication path to transmit the read instructions to from the control room to the bay audio speaker for output by the bay audio speaker;
wherein the electronic processing device is programmed to:
generate the instructions; and
display the instructions on a display device disposed in the control room to be read by the operator.

8. A communication method for communicating between an imaging bay containing a medical imaging device and a control room containing a controller for controlling the medical imaging device, the communication method comprising:
receiving bay audio from the imaging bay at the control room;
using an electronic processing device, modifying the bay audio to generate modified bay audio; and
presenting the modified bay audio in the control room.

9. The communication method of claim 8, further including:
receiving operator instructions from an operator in a first language; and
translating the operator instructions to generate the instructions in a second language that is different from the first language.

10. The communication system of claim 8, further including:
generating the instructions; and
synthesizing an audio signal representing the instructions which is transmitted from the control room to the bay audio speaker for output by the bay audio speaker.

11. A medical workflow assistance system for assisting with a medical workflow, the system comprising:
at least one microphone configured to acquire audio of an interaction between a patient whose is the subject of the medical workflow and a medical professional interacting with the patient during the medical workflow; and
an electronic processing device programmed to:
analyze the audio acquired by the at least one microphone to determine actionable information about the patient and/or the medical professional;
determine a modification of or addition to the medical workflow based on the actionable information; and
automatically implement the modification of or addition to the medical workflow.

12. The medical workflow assistance system of claim 11, wherein the at least one microphone is a component of a communication system for communicating between an imaging bay containing a medical imaging device and a control room containing a controller for controlling the medical imaging device, the medical workflow includes a medical imaging examination performed on the patient using the communication system comprising:
an intercom including:
a bay audio speaker disposed in the imaging bay;
a bay microphone disposed in the imaging bay; and
a communication path via which bay audio from the imaging bay acquired by the bay microphone is transmitted to the control room;
wherein the at least one microphone of the medical workflow assistance system comprises the bay microphone; and
wherein the electronic processing device is programmed to analyze the audio to determine the actionable information about the patient and/or the medical professional, determine the modification of or addition to the medical workflow comprising a modification of or addition to a workflow of the medical imaging examination based on the actionable information, and automatically implement the modification of the workflow of the medical imaging examination.

13. The medical workflow assistance system of claim 11, wherein:
the at least one microphone is a component of an audio or video call system, and the medical workflow includes a telehealth session conducted between the patient and the medical professional using the audio or video call system.

14. The medical workflow assistance system of claim 11, wherein:
the analysis of the audio acquired by the at least one microphone includes detecting the actionable information comprising a mental or physical condition of the patient from the audio; and
the determined modification of or addition to the medical workflow includes at least one of (i) recording an indication of the detected medical or physical condition of the patient in an electronic patient record of the patient, (ii) requesting assistance of a third party selected by the electronic processing device based on the detected medical or physical condition of the patient, (iii) communicating the indication of the detected medical or physical condition of the patient to the medical professional; and/or (iv) communicating the indication of the detected medical or physical condition of the patient to a medical doctor treating the patient.

15. The medical workflow assistance system of claim 14, wherein the detected mental or physical condition includes a respiratory condition of the patient detected by analyzing breathing of the patient in the audio acquired by the at least one microphone;
wherein the detected mental or physical condition includes frailty of the patient detected based on one or more of articulation rate, mean fundamental frequency, and/or voice intensity range of speech of the patient in the audio acquired by the at least one microphone.

16. The medical workflow assistance system of claim 11, wherein analysis of the audio acquired by the at least one microphone includes:
performing a natural language process (NLP) on the audio to extract the actionable information about the patient and/or the medical professional.

17. The medical workflow assistance system of claim 16, wherein the determined actionable information includes a medical condition of the patient identified based on words or phrases obtained by the NLP of speech of the patient in the audio.

18. The medical workflow assistance system of claim 11, wherein analysis of the audio acquired by the at least one microphone includes:
detecting one or more voice biomarkers of the patient, wherein the actionable information is actionable information about the patient determined from the one or more voice biomarkers.

19. The medical workflow assistance system of claim 11, wherein:
the analysis of the audio acquired by the at least one microphone includes detecting the actionable information comprising a mental or physical condition of the medical professional; and
the determined modification of or addition to the medical workflow includes at least one of (i) recording an indication of the detected medical or physical condition of the medical professional in an electronic employee record of the medical professional, (ii) requesting assistance of a third party selected by the electronic processing device based on the detected medical or physical condition of the medical professional, (iii) suspending the medical professional, and/or (iv) scheduling remedial training for the medical professional.

20. The medical workflow assistance system of claim 19, wherein the analysis includes performing voice analytics on speech of the medical professional in the audio to detect stress or exhaustion of the medical professional or to detect slurred speech of the medical professional, and the determined modification includes requesting assistance of a third party selected by the electronic processing device based on the detected slurred speech.
